(19) United States
(12) Patent Application Publication
(10) Pub. No.: US 2019/0304577 A1
(43) Pub. Date: Oct. 3, 2019

(54) COMMUNICATION VIOLATION SOLUTION

(71) Applicant: Konica Minolta Healthcare Americas, Inc., Wayne, NJ (US)

(72) Inventor: Hiroyuki Kubota, Wayne, NJ (US)

(73) Assignee: Konica Minolta Healthcare Americas, Inc., Wayne, NJ (US)

(21) Appl. No.: 15/944,396

(22) Filed: Apr. 3, 2018

(51) Int. Cl. G16H 10/60; G16H 15/00; H04L 29/08

(52) U.S. Cl. CPC G16H 10/60; H04L 67/12; G16H 15/00

(57) ABSTRACT

A management server that communicates with a plurality of medical devices includes a transceiver that receives from one of the plurality of medical devices a first medical file that comprises a request for a medical record; and a processor that: detects whether a format of the first medical file complies with a predetermined standard format; in response to detecting that the format of the first medical file does not comply with the predetermined standard format, converts the format of the first medical file to the predetermined standard format; handles the request based on the converted first medical file; generates a second medical file as a response to the request in the format of the first medical file; and transmits the response to the medical device.

### BACKGROUND

Electronic medical records, including medical images and other medical data play a crucial role in the diagnosis of patients. Healthcare facilities (e.g., hospitals) have realized the benefits of electronically storing medical records. The digitalization of medical images and other data not only enables users to easily access the medical images and medical data, but also enables the images and data to be easily shared between multiple healthcare facilities.

In the healthcare industry, the use of a system known as a Picture Archiving and Communications System (“PACS”) is becoming increasingly popular for convenient storage and access of medical images and reports. Generally, a PACS comprises a multitude of devices working cooperatively to digitally capture, store, manage, distribute, and display medical images generated by various imaging modalities, such as computed tomography (CT), magnetic resonance imaging (MRI), position emission tomography (PET), ultrasound, X-ray, etc. PACS allows various healthcare facilities to share all types of images and reports captured or created internally or externally.

To exchange medical data in the PACS, some industry standards are adopted, such as Digital Imaging and Communications in Medicine (DICOM) and Health Level Seven International (HL7). These standards are updated at least once a year to catch up on new technologies and trends in the medical industry.

In general, a management server or a user terminal of the PACS tends to be replaced with the new one that complies with the latest standard. On the other hand, an imaging device, such as CT and MRI, is unlikely to be replaced because of its replacement cost. Thus, it is common that an operating PACS includes a plurality of devices operating in different versions of standards, which requires software operating in the management server, the user terminal, and/or the imaging device be customized so that these devices can communicate with each other properly under the mixed environments.

### SUMMARY

In general, the invention relates to a medical imaging system.

In one aspect according to one or more embodiments of the invention, a management server that communicates with a plurality of medical devices includes a transceiver that receives from one of the plurality of medical devices a first medical file that comprises a request for a medical record; and a processor that: detects whether a format of the first medical file complies with a predetermined standard format; in response to detecting that the format of the first medical file does not comply with the predetermined standard format, converts the format of the first medical file to the predetermined standard format; handles the request based on the converted first medical file; generates a second medical file as a response to the request in the format of the first medical file; and transmits the response to the medical device (i.e., the one of the plurality of medical devices).

In another aspect according to one or more embodiments of the invention, a method for a management server that communicates with a plurality of medical devices includes receiving from one of the plurality of medical devices a first medical file that comprises a request for a medical record; detecting whether a format of the first medical file complies with a predetermined standard format; in response to detecting that the format of the first medical file does not comply with the predetermined standard format, converting the format of the first medical file to the predetermined standard format; handling the request based on the converted first medical file; generating a second medical file as a response to the request in the format of the first medical file; and transmitting the response to the medical device (i.e., the one of the plurality of medical devices).

In another aspect according to one or more embodiments of the invention, a non-transitory computer readable medium (CRM) storing instructions for a management server that communicates with a plurality of medical devices, the instructions include functionality for: receiving from one of the plurality of medical devices a first medical file that comprises a request for a medical record; detecting whether a format of the first medical file complies with a predetermined standard format; in response to detecting that the format of the first medical file does not comply with the predetermined standard format, converting the format of the first medical file to the predetermined standard format; handling the request based on the converted first medical file; generating a second medical file as a response to the request in the format of the first medical file; and transmitting the response to the medical device (i.e., the one of the plurality of medical devices).

Other aspects of the invention will be apparent from the following description and the appended claims.

### BRIEF DESCRIPTION OF DRAWINGS

FIG. 1 shows a medical system in accordance with one or more embodiments of the invention.

FIG. 2 shows a block diagram of a medical system in accordance with one or more embodiments of the invention.

FIG. 3 shows a flowchart in accordance with one or more embodiments of the invention.

FIGS. 4-8 each show an example of convert operations in accordance with one or more embodiments of the invention.

FIG. 9 shows a computer system in accordance with one or more embodiments of the invention.

### DETAILED DESCRIPTION

Specific embodiments of the invention will now be described in detail with reference to the accompanying figures. Like elements in the various figures are denoted by like reference numerals for consistency.

In the following detailed description of embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers does not imply or create a particular ordering of the elements or limit any element to being only a single element unless expressly disclosed, such as by the use of the terms “before,” “after,” “single,” and other such terminology. Rather, the use of ordinal numbers is to distinguish between the elements. By way of an example, a first element is distinct from a second element, and the first element may encompass more than one element and succeed (or precede) the second element in an ordering of elements.

It is to be understood that one or more of the steps shown in the flowcharts may be omitted, repeated, and/or performed in a different order than the order shown. Accordingly, the scope of the invention should not be considered limited to the specific arrangement of steps shown in the flowcharts.

In the following detailed description of embodiments of the invention, numerous specific details are set forth to provide a more thorough understanding of the invention. However, it will be apparent to one of ordinary skill in the art that the invention may be practiced without these specific details. In other instances, well-known features have not been described in detail to avoid unnecessarily complicating the description.

In general, one or more embodiments of the invention provide a management server, a method, and a non-transitory computer readable medium for storing and managing electronic medical records in a medical system such as a PACS in which a plurality of medical devices operating in different versions of standards such as “DICOM 2000” and “DICOM 2017” or different implementations of the same version. The management server of the medical system in accordance with one or more embodiments of the invention enables various kinds of medical devices, such as medical imaging devices, medical user terminals, and other management servers to be integrated into a single system even when these medical devices support different versions or implementations of the standard that are not fully compatible. For example, the management server in accordance with one or more embodiments of the invention not only corrects the format of a medical file that is received from the medical device and is incompatible with the standard format being used in the management server, but coverts the format of the standard format that is transmitted from the management server to the other format so that the management server and the medical devices can exchange a medical record correctly. As a result, even when a system update is made in a medical facility except some of the medial devices such as expensive imaging devices, the management server eliminates the need for modifying software operating in the medical devices to maintain compatibility.

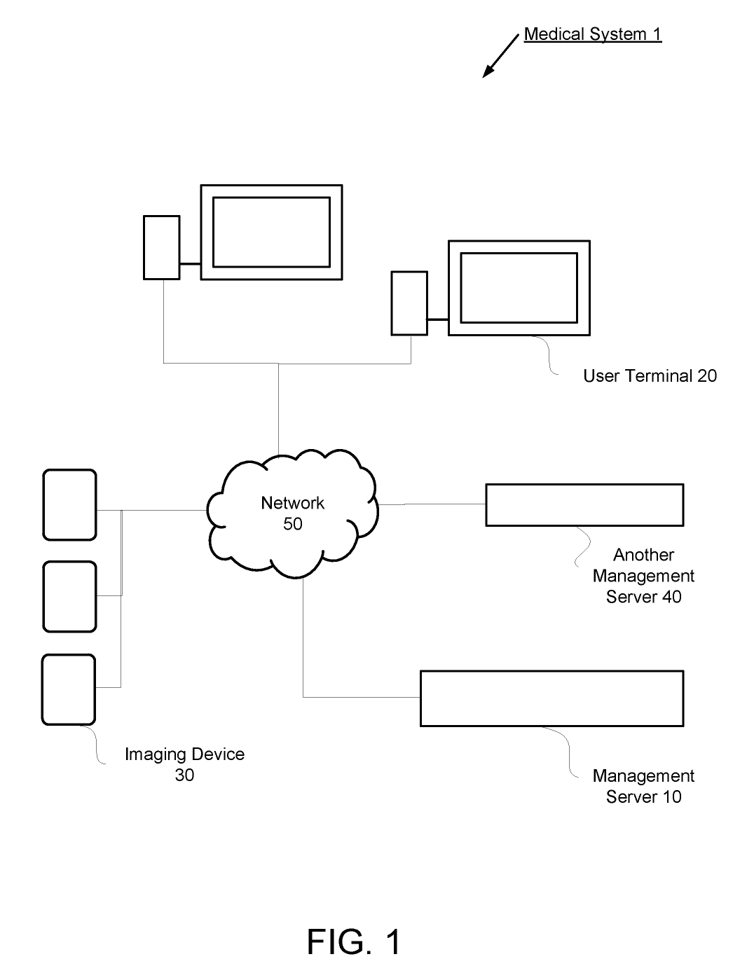

FIG. 1 shows a medical system 1 in accordance with one or more embodiments of the invention. The medical system 1 includes a management server (hereinafter “first management server”) 10, a user terminal 20, an imaging device 30, and another management server (hereinafter “second management server”) 40. These medical devices are connected via a network 50 and installed in a medical facility or over a plurality of medical facilities.

In one or more embodiments of the invention, the medical system is a PACS. The first management server 10 and the other medical devices 20, 30, and 40 exchange a medical record with a predetermined medical data format, such as a Digital Imaging and Communications in Medicine (DICOM).

For example, a DICOM file contains a request (or command) to retrieve a list of medical records (e.g., medical images, medical image orders, medical image interpretation reports, or medical examination reports) or store or retrieve the medical record itself in the first management server 10. The DICOM file may comprise a set of: (i) an attribute or element tag (hereinafter “a tag,” which indicates an attribute of the medical record; (ii) one or more values for the attribute; and (iii) a value representation (VR) that describes the data type and format of the value. Such attributes include, but are not limited to, a patient's name, a patient ID, a patient's birth date, a patient's sex, and a study ID. The DICOM standard defines the tag that identifies an attribute by a pair of four hexadecimal digit numbers (e.g., “0010,0010” for a patient name).

In one or more embodiments of the invention, the first management server 10 receives from the other medical devices a request for retrieving a list of the medical records stored in the first management server 10 and the medical record itself, and for storing the medical record in the first management server 10. The request is made in the form of a DICOM file. Thus, the DICOM file may include the command for a request, a medical record, and a set of at least a tag and value for the medical record. The first management server 10 may accept and respond to the request in which the format of the DICOM file (e.g., the number of tags, the format of values, the data type of the values, etc.) does not comply with the DICOM standard being used in the first management server 10. The first management server 10 may detect such inconsistency, convert the format of the received DICOM file to the server's DICOM format, and handle the request properly. When sending a response to the other medical device, the first management server 10 transmits a DICOM file in the format that is being used by the medical device. Thus, in case the format used by the other medical device does not comply with the standard, the first management server 10 transmits the response by using such a non-compliant format.

The user terminal 20 in accordance with one or more embodiments of the invention is a medical user terminal operated by a medical person or user (hereinafter “user”). The user terminal 20 has a display and an input device so that the user can control the user terminal 20 to retrieve a list of the medical records stored in the first management server 10 or to retrieve the medical record itself by specifying attributes. In one or more embodiments of the invention, the user terminal transmits a request for retrieving the medical record in the form of a DICOM file, which complies with a certain version or implementation of the DICOM standard that is not fully compatible with the DICOM standard being used in the first management server 10.

The imaging device 30 in accordance with one or more embodiments of the invention is an imaging modality, such as Computed Radiography (CR), CT, and MRI. In addition to such imaging function, the imaging device 30 is capable of accepting an input from the user and communicating with the first management server 10 for storing in the first management server 10 a medical record such as a medical image or retrieve a medical record such as a medical imaging order stored in the first management server 10 by specifying attributes. Similar to the user terminal 20, the imaging device 30 transmits a request for storing or retrieving the medical record in the form of a DICOM file, which complies with a certain version or implementation of the DICOM standard that is not fully compatible with the DICOM format being used in the first management server 10.

The other (second) management server 40 in accordance with one or more embodiments of the invention may transmit to the first management server 10 a request for storing a medical record or retrieving the medical records stored in the first management server 10 in the form of the DICOM file. The other management server 40 may also use a certain version or implementation of the DICOM standard that is not fully compatible with the DICOM format being used in the first management server 10.

In one or more embodiments of the invention, the network 50 is formed by one or more networks including an intranet deployed in a medical facility and/or the Internet.

According to this configuration, the first management server 10 allows various kinds of existing medical devices that use an incompatible medical data format to be integrated into a system without modifying existing software or implementing any additional software for each of the medical devices.

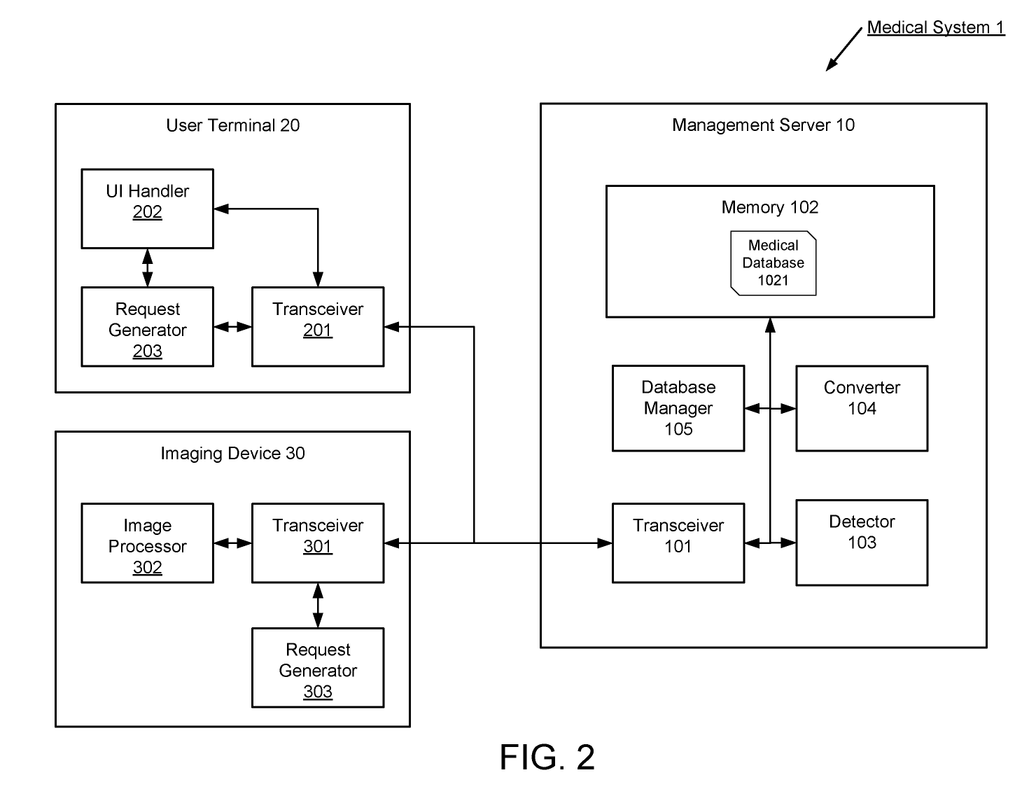

FIG. 2 shows a block diagram of a medical system in accordance with one or more embodiments of the invention. The following description is set forth on the assumption that the medical system uses one or more different DICOM formats for exchanging medical records.

As shown in FIG. 2, the first management server 10 of the medical system 1 includes a transceiver 101, a memory 102, a detector 103, a converter 104, and a database manager 105.

The transceiver 101 of the first management server 10 communicates with another medical device such as the user terminal 20 and the imaging device 30. The transceiver 101 receives a request from such a medical device in the form of a DICOM file, and transmits a response to the medical device. The response includes, but is not limited to, a simple acknowledgement that indicates the success of the communication and a response in the form of a DICOM file based on a specific request for retrieving a medical record including a medical image, a medical image order, a medical image interpretation report, and a medical examination report.

The memory 102 of the first management server 10 stores medical database 1021 for managing medical records in a manner that complies with a certain version or implementation of the DICOM standard. In one or more embodiments of the invention, each of the medical report comprises a plurality of sets of an attribute (e.g., a patient ID, a patient name, a date of birth, etc.), at least one value, and a VR (i.e., data type and format for the values). As stated above, each of the attributes is specified by a predetermined tag, and the supported tags and the format for the values depend on versions or implementations of the DICOM standard.

When the transceiver 101 receives a request from a medical device in the form of the DICOM file, the detector 103 of the first management server 10 parses the DICOM file and detects whether a format of the DICOM file complies with the standard (e.g., the format used by the first management server 10). To detect the violation, the detector 103 may refer to a format database that defines correct format of the DICOM standard used by the first management server 10. For example, the detector 103 detects the format incompliance by comparing the format of the received DICOM file (e.g., the total number of tags, the digit or character format of the value, etc.) with the standard format read from the format database.

In one or more embodiments of the invention, the detector 103 detects that the format of the DICOM file (Format A) does not comply with the standard format (Format B) if:

(1) Format A lacks a tag, which is required by Format B;

(2) Format A contains a tag, which is not expected by Format B;

(3) The digit value for an attribute in Format A is not allowed in Format B;

(4) The value of an attribute in Format A follows or is followed by an unnecessary space character, which is not expected by Format B;

(5) The total number of values required for an attribute (i.e., VM number) in Format A is not consistent with the one defined in Format B;

(6) The length of the value for an attribute in Format A is not consistent with the one defined in Format B;

(7) The format of a value for a predetermined attribute (e.g., patient's name, date and time, and age) in Format A is not consistent with the one defined in Format B;

(8) The data value of an attribute in Format A is empty or invalid in Format B;

(9) The character set and encoding of the value for an attribute in Format A is not consistent with the one defined in Format B; and

(10) The VR for a certain attribute in Format B is not consistent with the one defined in Forma B.

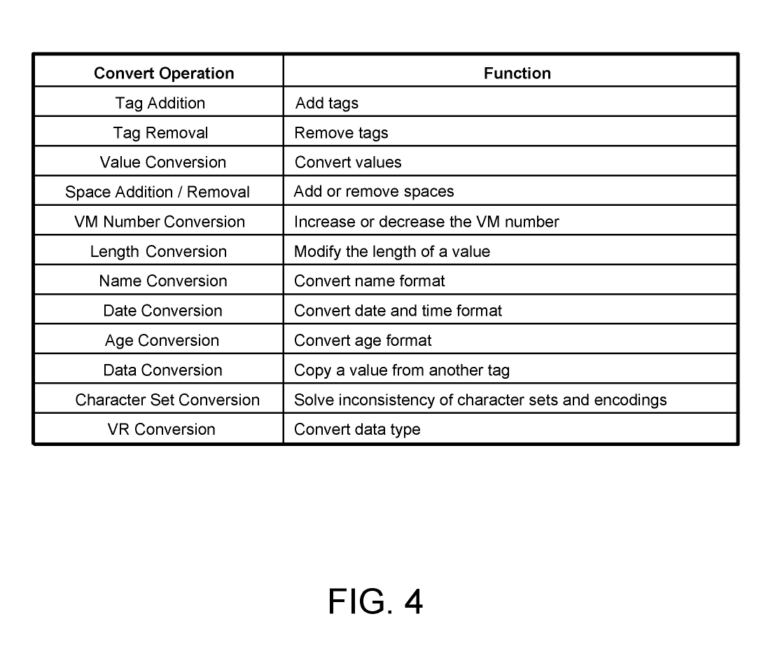

When the detector 103 has detected that the format of the DICOM file received from the other medical device does not comply with the standard format being used in the first management server 10, the converter 104 converts the format of the received file to the standard format. In one or more embodiments of the invention, as shown in FIG. 4, the converter 104 applies the following convert operations to the received DICOM file (Format A) so that the received file complies with the standard format (Format B):

(1) Add a tag, which is required by Format B;

(2) Remove a tag, which is not expected by Format B;

(3) Convert the digit value for an attribute according to Format B;

(4) Remove an unnecessary space character following or is followed by the value of an attribute, which is not expected by Format B;

(5) Increase or decrease the total number of values required for an attribute (i.e., VM number) according to Format B;

(6) Modify the length of the value for an attribute according to Format B;

(7) Convert the format of a value for a predetermined attribute (e.g., patient's name, date and time, and age) according to Format B;

(8) Add a correct data value for an attribute according to Format B;

(9) Convert the character set and encoding of the value for an attribute according to Format B; and

(10) Convert the VR for an attribute according to Format B.

Additionally, once the first management server 10 has handled the request from the other medical device (e.g., store or retrieve a medical record), the detector 103 generates an intermediate DICOM file for response that complies with the standard format being used in the first management server 10, and then converts the intermediate DICOM file to the format originally used by the other medical device. In one or more embodiments of the invention, the converter 104 applies the following (reverse) convert operations to the intermediate DICOM file for response (Format B) based on the original format of the received DICOM file (Format A):

(1) Remove a tag, which is required by Format B but not required by Format A;

(2) Add a tag, which is expected by Format B but not expected by Format A;

(3) Convert the digit value for an attribute according to Format A;

(4) Add a space character following or is followed by the value of an attribute, which is expected by Format A;

(5) Decrease or increase the total number of values required for an attribute (i.e., VM number) according to Format A;

(6) Modify the length of the value for an attribute according to Format A;

(7) Convert the format of a value for a predetermined attribute (e.g., patient's name, date and time, and age) according to Format A;

(8) Convert the data value of an attribute according to Format A;

(9) Convert the character set and encoding of the value for an attribute according to Format A; and

(10) Convert the VR for an attribute according to Format A.

The database manager 105 of the first management server 10 handles a query to the medical database 1021 and generates the search result. In one or more embodiments of the invention, the database manager 105 searches and retrieves the medical records in the medical database 1021 based on the request (i.e., the received DICOM file) from the medical device. As discussed above, the retrieved result is transmitted to the medical device as the form of a DICOM file, after being converted back to the original format.

The user terminal 20 of the medical system 1 in accordance with one or more embodiments of the invention includes a transceiver 201, a UI handler 202, and a request generator 203.

The transceiver 201 of the user terminal 20 communicates with the first management server 10. In one or more embodiments of the invention, the transceiver 201 transmits a request for retrieving a medical record in the form of a DICOM file, which, however, does not comply or fully compatible with the DICOM format used in the first management server 10. On the contrary, the transceiver 201 receives a response (i.e., the requested medical record) from the first management server 10 in the form of the DICOM file, the format of which is originally used to transmit the request.

The UI handler 202 of the user terminal 20 handles input from and output to a user interface, such as a graphical user interface. For example, the UI handler 202 enables a user of the user terminal 20 to specify a medical record by attributes and their values (e.g., a study ID, a patient ID, patient's sex, a modality, a study date, a patient name, and a date of birth), and view the medical record on the screen.

The request generator 203 of the user terminal 20 generates a request to be transmitted to the first management server 10 based on user input made via the UI handler 202. In one or more embodiments of the invention, once the attributes and values have been specified via the UI handler 202, the request generator 203 generates a request for retrieving a medical record based on the specified attributes and values in the form of a DICOM file.

The imaging device 30 of the medical system 1 in accordance with one or more embodiments of the invention includes a transceiver 301, an image processor 302, and a request generator 303.

The transceiver 301 of the imaging device 30 communicates with the first management server 10. In one or more embodiments of the invention, the transceiver 301 transmits a request for storing a medical record such as a medical image in the first management server 10 or for retrieving a medical record such as a medical image order in the form of a DICOM file, which, however, does not comply or fully compatible with the DICOM format used in the first management server 10. Additionally, the transceiver 301 receives a response from the first management server 10 in the form of the DICOM file, the format of which is originally used to transmit the request.

The image processor 302 of the imaging device 30 takes medical images of a patient and generates digital data encoded with a common image format such as JPEG.

The request generator 303 of the imaging device 30 generates a request for storing or retrieving a medical record in response to the operation of an imaging device user. In one or more embodiments of the invention, the request generator 303 generates such a request in the form of a DICOM file.

While FIG. 2 shows one particular configuration of components for illustration purposes, other configurations may be used without departing from the scope of the invention. For example, various components may be combined to form a single component. As another example, the functionality performed by a single component may be performed by two or more components.

Each of the components 102-105 may be located on the same first management server 10 or may be located on different computing devices connected by the network 50 having wired and/or wireless segments. Further, one or more of the components 101-105, 201-203, and 301-303 may be implemented by one or more processors or other dedicated hardware.

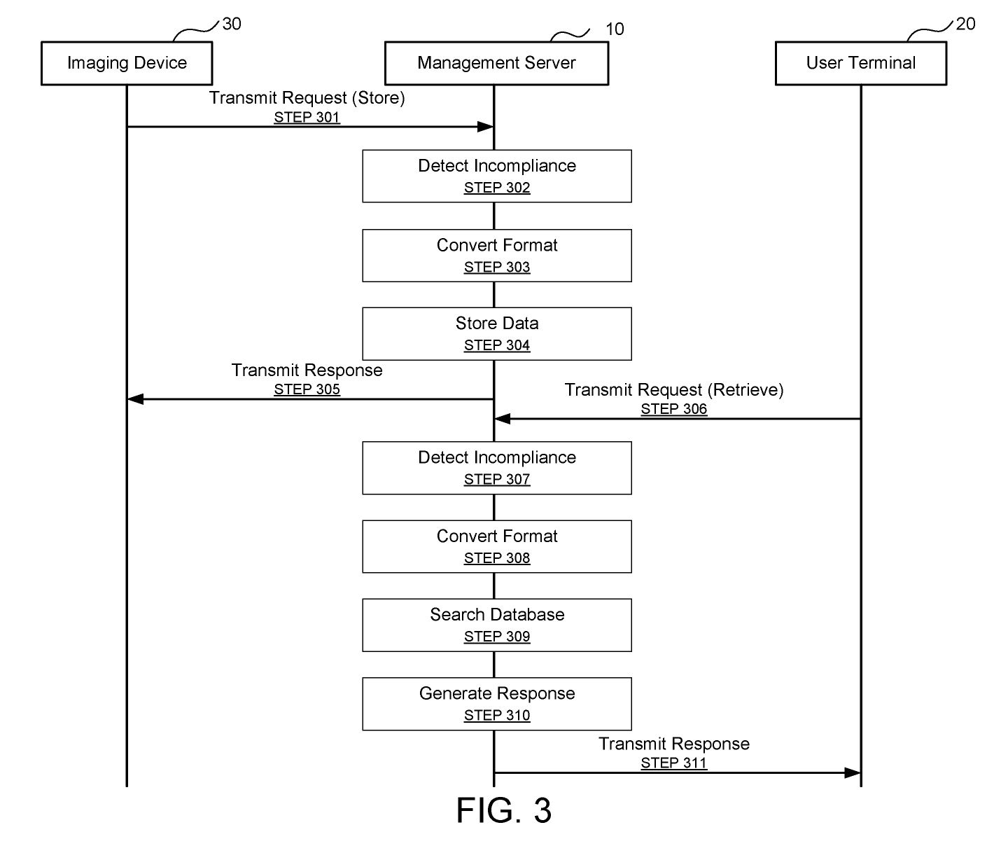

FIG. 3 shows a flowchart in accordance with one or more embodiments of the invention. The process depicted in FIG. 3 may be used for the imaging device 30 to transmit a request for storing a medical image and for the user terminal 20 to retrieve the medical image. In the example of FIG. 3, the imaging device 30 and the user terminal 20 each transmit a request for storing a medical image and a medical image order using the version of a DICOM file, which does not comply or fully compatible with the DICOM standard format being used in the first management server 10.

One or more of the steps in FIG. 3 may be performed by the components of the medical system 1, discussed above with reference to FIG. 2. In one or more embodiments of the invention, one or more of the steps shown in FIG. 3 may be omitted, repeated, and/or performed in a different order than the order shown in FIG. 3. Accordingly, the scope of the invention is not limited to the specific arrangement of steps shown in FIG. 3.

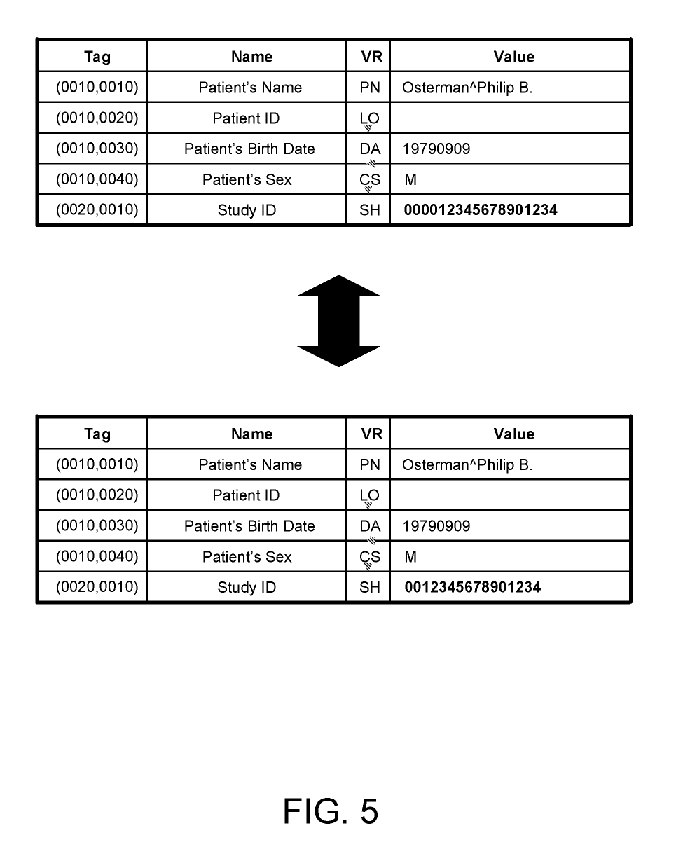

Initially, in response to user's operation, the request generator 303 of the imaging device 30 generates a request for storing a medical record such as a medical image and its attributes in the form of a DICOM file, and the transceiver 301 transmits the generated DICOM file to the first management server 10 via the network 50 (S301). In this example, the request generator 203 generates a DICOM file including a request command (e.g., C-STORE) for storing a medical image content and related attributes and values, such as a patient name, a patient ID, a patient's birth date, a patient's sex, and a study ID, as shown in FIG. 5. Any other attributes defined in the DICOM standard may be contained in the DICOM file. As discussed above, the attributes and the values are described in the form of a set of a tag, one or more values, and the VR (data type and format).

When the transceiver 101 of the first management server 10 receives the request (i.e., the DICOM file) from the imaging device 30, the detector 103 of the first management server 10 parses the DICOM file and detects whether a format of the DICOM file complies with the standard used by the first management server 10 (S302). For example, the detector 103 refers to a format database that defines the correct format of the DICOM standard being used in the first management server 10, and detects format incompliance by comparing the format of the received DICOM file with the standard format read from the format database.

When the detector 103 detects that the received DICOM file does not comply with the standard format being used in the first management server 10, the converter 104 converts the received DICOM file to the standard format (S303).

FIGS. 5-8 each show an example of the incompliance detection in S302 and the format conversion in S303. Each of the figures includes two tables with a plurality of sets of a tag, a VR, and a value included in a DICOM file. Each table further includes a name column, which is added for the purpose of explanation, and may not be included in the DICOM file. The upper table indicates the attributes and values included in the DICOM file generated by a medical device (e.g., the user terminal 20 or the imaging device 30), and the lower table indicates the ones converted to the standard format being used in the first management server 10.

In the example of FIG. 5, the detector 103 detects that the digit value for the “Study ID” attribute does not comply with the standard, which requires the value be sixteen-digit numbers. Because the value for the “Study ID” attribute in the DICOM file from the medical device has eighteen-digit numbers, the converter 104 of the first management server 10 converts the digit value by removing the first two digits “00.”

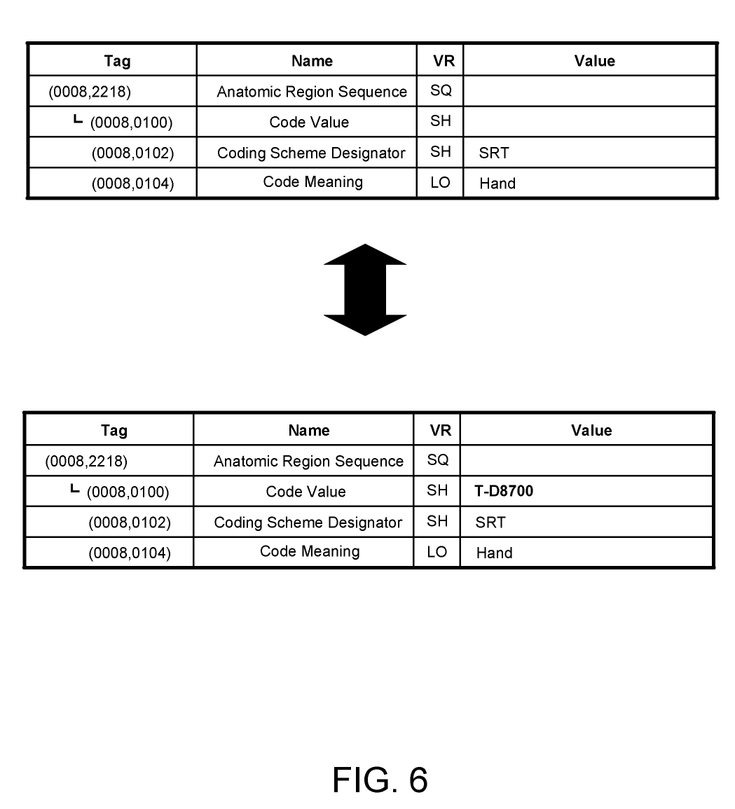

In the example of FIG. 6, the detector 103 detects that the value for the “Code Value” attribute does not comply with the standard, which does not allow the field to be empty. Because the “Code Value” can be retrieved based on the value of another attribute “Code Meaning,” the converter 104 looks up the term “Hand” in an external dictionary, and adds the identified value “T-D8700” to the “Code Value.” The external dictionary may be stored in the memory 102 of the first management server 10 in advance or any device that is accessible from the first management server 10 via the network 50.

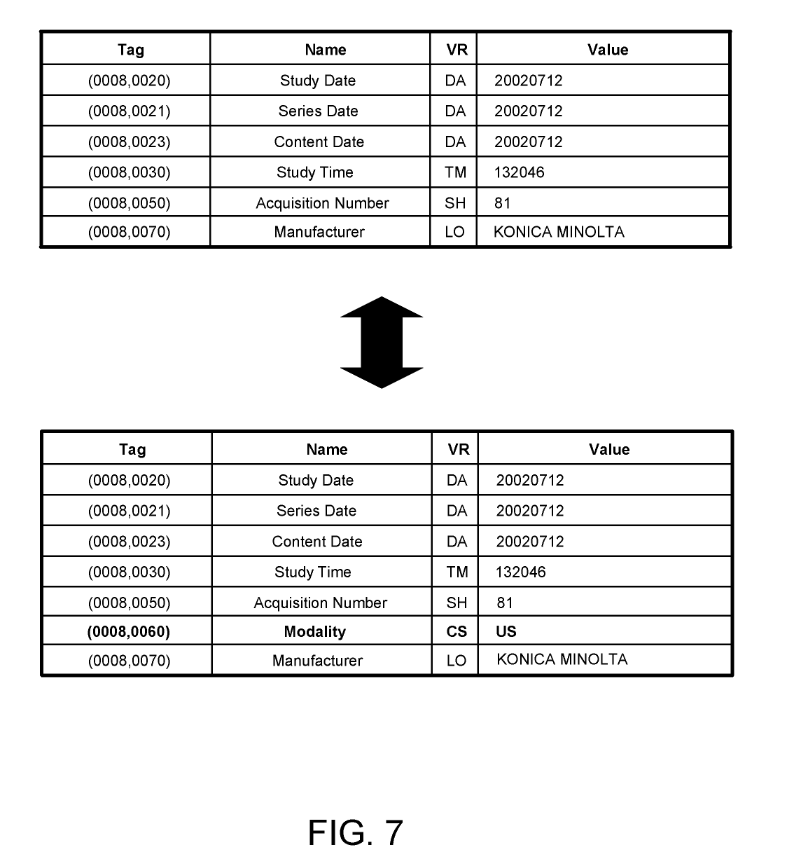

In the example of FIG. 7, the detector 103 detects that the DICOM file from the medical device lacks the “Modality” attribute, which is required by the standard. Thus, the converter 104 insert the “Modality” attribute with a predetermined default value “US.”

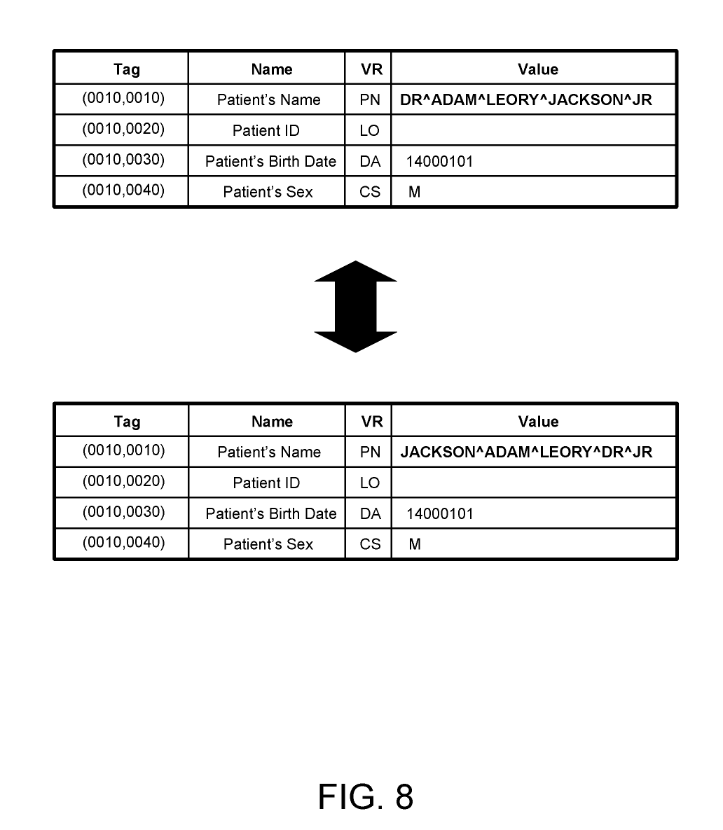

In the example of FIG. 8, the detector 103 detects that the format of the value for the “Patient's Name” attribute does not comply with the standard, which requires the order of the “Patient's Name” be “Family Name,” “Given Name,” “Middle Name,” “Prefix,” and “Suffix.” Because the format used by the imaging device 30 deviates from the standard, the converter 104 rearranges the order. The converter 104 may any known scheme to parse, recognize, and rearrange these portions of the name.

Referring back to FIG. 3, once the converter 104 has completed the convert operation, the database manager 105 stores in the medical database 1021 the medical record (e.g., the medical image) using the converted DICOM file (S304). Finally, the transceiver 101 of the management server 10 transmits an acknowledgment as a response to the imaging device 30 (S305).

After S301-305, in response to user input, the request generator 203 of the user terminal 20 generates a request for retrieving a medical record such as the medical image that has been transmitted from the imaging device 30, and the transceiver 201 transmits the request to the first management server 10 (S306). For example, the request may be formed according to the DICOM file format, including a command for retrieving the medical images as well as a plurality of sets of an attribute, a VR, and at least one value.

When the transceiver 101 of the first management server 10 receives the request from the user terminal 20, similar to S302, the detector 103 parses the DICOM file and detects whether the format of the DICOM file complies with the standard being used in the first management server 10 (S307). Next, similar to S303, when the detector 103 detects that the received DICOM file does not comply with the DICOM standard, the converter 104 converts the format of the received DICOM file according to the DICOM standard (S308) so that the medical record may be looked up and retrieved from the medical database 102. The database manager 105 then searches the medical database 1021 to locate the requested medical record based on the converted DICOM file (S309).

Once the requested medical record has been retrieved, the converter 104 generates a new DICOM file as a response to the request from the user terminal 20 (S310). For example, the new DICOM file is generated by converting an intermediate DICOM file including the requested medical record under the standard being used in the first management server 10 to the format being used by the user terminal 20. In other words, the converter 104 executes reverse conversion on the intermediate DICOM file generated in the standard format being used in the first management server 10. For example, assuming that the user terminal 20 uses the format shown in the upper table of FIG. 5, the converter 104 first generates an intermediate DICOM file according to the DICOM standard as shown in the lower table of FIG. 5, and then converts the intermediate DICOM file to the format used by the user terminal 20, as shown in the upper table of FIG. 5. To execute this inverse operation, the converter 104 may store in the memory 102 the information about the convert operation executed at S308. Finally, the transceiver 101 transmits the converted DICOM file to the user terminal 20 as a response (S311).

According to this configuration, the first management server 10 can not only accept a medical file from an existing medical device that operates in an incompatible version of the standard but also transmit the medical file to such an old medical device without the need for any software modification.

Embodiments of the invention may be implemented on virtually any type of management server and user terminal, regardless of the platform being used. For example, the first management server 10 may be one or more desktop computers, servers, blades in a server chassis, or any other type of computing device or devices that includes at least the minimum processing power, memory, and input and output devices to perform one or more embodiments of the invention. Additionally, the user terminal 20 may be one or more mobile devices (e.g., laptop computer, smartphone, personal digital assistant, tablet, or other mobile device), desktop computers, servers, blades in a server chassis, or any other type of computing device or devices that includes at least the minimum processing power, memory, and input and output devices to perform one or more embodiments of the invention.

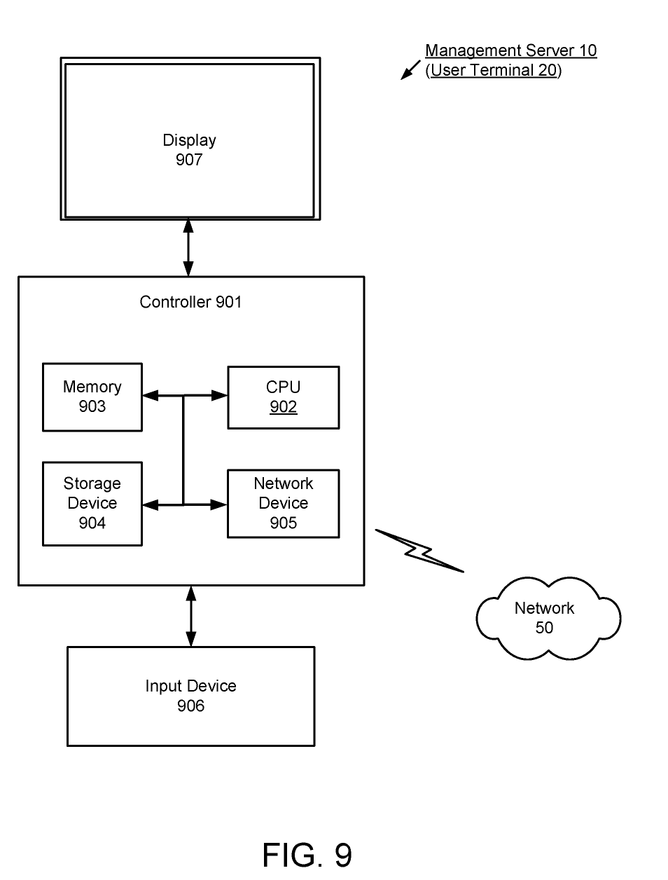

For example, as shown in FIG. 9, the first management server 10 and the user terminal 20 may include one or more CPUs 902, associated memory 903 (e.g., random access memory (RAM), cache memory, flash memory, etc.), one or more storage devices 904 (e.g., a hard disk, a solid state drive, an optical drive such as a compact disk (CD) drive or digital versatile disk (DVD) drive, a flash memory drive, etc.), a network device 905 (e.g., a network interface card, a wireless LAN module, a wide area network module, a Bluetooth module, a ZigBee module, an infra-red communication module, etc.), and numerous other elements and functionalities.

The CPU 902 may be an integrated circuit for processing instructions. For example, the computer processor may be one or more cores or micro-cores of a processor. The CPU 902 may have one or more caches which are faster memories than the (main) memory 903. The first management server 10 and the user terminal 20 may also include one or more input devices 906, such as a keyboard and a mouse or any other type of input device. Further, the first management server 10 and the user terminal 20 may include a display 907. The first management server 10 and the user terminal 20 may also include a network device 905 connected to the network 50 (e.g., a local area network (LAN), a wide area network (WAN) such as the Internet, mobile network, or any other type of network) via a network interface connection (not shown). The input and output devices may be locally or remotely (e.g., via the network 50) connected to the CPU 902, memory 903, storage device 904, and network device 905. Many different types of computing systems exist, and the aforementioned input and output devices may take other forms.

Software instructions in the form of computer readable program code to perform embodiments of the invention may be stored, in whole or in part, temporarily or permanently, on a non-transitory computer readable medium such as a CD, DVD, Blu-ray Disc, storage device, a diskette, a tape, flash memory, physical memory, or any other computer readable storage medium. Specifically, the software instructions may correspond to computer readable program code that, when executed by a processor, is configured to perform embodiments of the invention.

Further, one or more elements of the aforementioned first management server 10 may be located at a remote location and connected to the other elements over a network 50. Further, one or more embodiments of the invention may be implemented on a distributed system having a plurality of nodes, where each portion of the invention may be located on a different node within the distributed system. In one embodiment of the invention, the node corresponds to a distinct computing device. Alternatively, the node may correspond to a computer processor with associated physical memory. The node may alternatively correspond to a computer processor or micro-core of a computer processor with shared memory and/or resources.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A management server that communicates with a plurality of medical devices, comprising:
a transceiver that receives from one of the plurality of medical devices a first medical file that comprises a request for a medical record; and
a processor that:
detects whether a format of the first medical file complies with a predetermined standard format;
in response to detecting that the format of the first medical file does not comply with the predetermined standard format, converts the format of the first medical file to the predetermined standard format;
handles the request based on the converted first medical file;
generates a second medical file as a response to the request in the format of the first medical file; and
transmits the response to the medical device.

2. The management server of claim 1, wherein
the processor generates the second medical file by converting a third medical file that complies with the predetermined standard format to the format of the first medical file.

3. The management server of claim 2, wherein
each of the first, the second, and the third medical file comprises a plurality of sets of an attribute and at least one value.

4. The management server of claim 3, wherein
the processor converts the third medical file to the format of the first medical file by adding or removing a set of the attribute and the value.

5. The management server of claim 3, wherein
the processor converts the third medical file to the format of the first medical file by converting the value.

6. The management server of claim 3, wherein
the processor converts the third medical file to the format of the first medical file by adding a space to or removing a space from the value.

7. The management server of claim 3, wherein
one of the plurality of sets includes one or more values, and
the processor converts the third medical file to the format of the first medical file by increasing or decreasing a total number of the one or more values.

8. The management server of claim 3, wherein
the processor converts the third medical file to the format of the first medical file by modifying a length of the value.

9. The management server of claim 3, wherein
the value indicates a name, a date and time, or an age; and
the processor converts the third medical file to the format of the first medical file by converting a format of the name, the date and time, or the age.

10. The management server of claim 3, wherein
the processor converts the third medical file to the format of the first medical file by copying a value of a first set of the plurality of sets to a second set of the plurality of sets.

11. The management server of claim 3, wherein
the processor converts the third medical file to the format of the first medical file by converting a character set and encoding of the value.

12. The management server of claim 3, wherein
the processor converts the third medical file to the format of the first medical file by converting a data type of the value.

13. The management server of claim 1, wherein
the request is made to store or retrieve a medical image, a medical image order, a medical image interpretation report, and a medical examination report.

14. The management server of claim 1, wherein
the first and the second medical file are formed by different versions of Digital Imaging and Communication in Medicine (DICOM) or Health Level Seven International (HL7) standards.

15. The management server of claim 1, wherein
the medical device is one of: a medical imaging device, a medical user terminal, and another management server that communicates with the plurality of medical devices used in a plurality of medical facilities.

16. A method for a management server that communicates with a plurality of medical devices, comprising:
receiving from one of the plurality of medical devices a first medical file that comprises a request for a medical record;
detecting whether a format of the first medical file complies with a predetermined standard format;
in response to detecting that the format of the first medical file does not comply with the predetermined standard format, converting the format of the first medical file to the predetermined standard format;
handling the request based on the converted first medical file;
generating a second medical file as a response to the request in the format of the first medical file; and
transmitting the response to the medical device.

17. The method of claim 16, wherein
the generating comprises generating the second medical file by converting a third medical file that complies with the predetermined standard format to the format of the first medical file.

18. The method of claim 17, wherein
each of the first, the second, and the third medical file comprises a plurality of sets of an attribute and at least one value.

19. The method of claim 16, wherein
the converting comprises converting the third medical file to the format of the first medical file by adding or removing a set of the attribute and the value.

20. A non-transitory computer readable medium (CRM) storing instructions for a management server that communicates with a plurality of medical devices, the instructions comprising functionality for:
receiving from one of the plurality of medical devices a first medical file that comprises a request for a medical record;
detecting whether a format of the first medical file complies with a predetermined standard format;
in response to detecting that the format of the first medical file does not comply with the predetermined standard format, converting the format of the first medical file to the predetermined standard format;
handling the request based on the converted first medical file;
generating a second medical file as a response to the request in the format of the first medical file; and
transmitting the response to the medical device.
